SEQUENCE LISTING (48 sequences)

SEQ ID NO: 1 - protein, 5 aa, Artificial
  synthetic peptide
DDDDD

SEQ ID NO: 2 - protein, 5 aa, Artificial
  synthetic peptide
DDDDE

SEQ ID NO: 3 - protein, 5 aa, Artificial
  synthetic peptide
DDDED

SEQ ID NO: 4 - protein, 5 aa, Artificial
  synthetic peptide
DDDEE

SEQ ID NO: 5 - protein, 5 aa, Artificial
  synthetic peptide
DDEDD

SEQ ID NO: 6 - protein, 5 aa, Artificial
  synthetic peptide
DDEDE

SEQ ID NO: 7 - protein, 5 aa, Artificial
  synthetic peptide
DDEED

SEQ ID NO: 8 - protein, 5 aa, Artificial
  synthetic peptide
DDEEE

SEQ ID NO: 9 - protein, 5 aa, Artificial
  synthetic peptide
DEDDD

SEQ ID NO: 10 - protein, 5 aa, Artificial
  synthetic peptide
DEDDE

SEQ ID NO: 11 - protein, 5 aa, Artificial
  synthetic peptide
DEDED

SEQ ID NO: 12 - protein, 5 aa, Artificial
  synthetic peptide
DEDEE

SEQ ID NO: 13 - protein, 5 aa, Artificial
  synthetic peptide
DEEDD

SEQ ID NO: 14 - protein, 5 aa, Artificial
  synthetic peptide
DEEDE

SEQ ID NO: 15 - protein, 5 aa, Artificial
  synthetic peptide
DEEED

SEQ ID NO: 16 - protein, 5 aa, Artificial
  synthetic peptide
DEEEE

SEQ ID NO: 17 - protein, 5 aa, Artificial
  synthetic peptide
EDDDD

SEQ ID NO: 18 - protein, 5 aa, Artificial
  synthetic peptide
EDDDE

SEQ ID NO: 19 - protein, 5 aa, Artificial
  synthetic peptide
EDDED

SEQ ID NO: 20 - protein, 5 aa, Artificial
  synthetic peptide
EDDEE

SEQ ID NO: 21 - protein, 5 aa, Artificial
  synthetic peptide
EDEDD

SEQ ID NO: 22 - protein, 5 aa, Artificial
  synthetic peptide
EDEDE

SEQ ID NO: 23 - protein, 5 aa, Artificial
  synthetic peptide
EDEED

SEQ ID NO: 24 - protein, 5 aa, Artificial
  synthetic peptide
EDEEE

SEQ ID NO: 25 - protein, 5 aa, Artificial
  synthetic peptide
EEDDD

SEQ ID NO: 26 - protein, 5 aa, Artificial
  synthetic peptide
EEDDE

SEQ ID NO: 27 - protein, 5 aa, Artificial
  synthetic peptide
EEDED

SEQ ID NO: 28 - protein, 5 aa, Artificial
  synthetic peptide
EEDEE

SEQ ID NO: 29 - protein, 5 aa, Artificial
  synthetic peptide
EEEDD

SEQ ID NO: 30 - protein, 5 aa, Artificial
  synthetic peptide
EEEDE

SEQ ID NO: 31 - protein, 5 aa, Artificial
  synthetic peptide
EEEED

SEQ ID NO: 32 - protein, 5 aa, Artificial
  synthetic peptide
EEEEE

SEQ ID NO: 33 - protein, 4 aa, Artificial
  synthetic peptide
DDDD

SEQ ID NO: 34 - protein, 4 aa, Artificial
  synthetic peptide
DDDE

SEQ ID NO: 35 - protein, 4 aa, Artificial
  synthetic peptide
DDED

SEQ ID NO: 36 - protein, 4 aa, Artificial
  synthetic peptide
DDEE

SEQ ID NO: 37 - protein, 4 aa, Artificial
  synthetic peptide
DEDD

SEQ ID NO: 38 - protein, 4 aa, Artificial
  synthetic peptide
DEDE

SEQ ID NO: 39 - protein, 4 aa, Artificial
  synthetic peptide
DEED

SEQ ID NO: 40 - protein, 4 aa, Artificial
  synthetic peptide
DEEE

SEQ ID NO: 41 - protein, 4 aa, Artificial
  synthetic peptide
EDDD

SEQ ID NO: 42 - protein, 4 aa, Artificial
  synthetic peptide
EDDE

SEQ ID NO: 43 - protein, 4 aa, Artificial
  synthetic peptide
EDED

SEQ ID NO: 44 - protein, 4 aa, Artificial
  synthetic peptide
EDEE

SEQ ID NO: 45 - protein, 4 aa, Artificial
  synthetic peptide
EEDD

SEQ ID NO: 46 - protein, 4 aa, Artificial
  synthetic peptide
EEDE

SEQ ID NO: 47 - protein, 4 aa, Artificial
  synthetic peptide
EEED

SEQ ID NO: 48 - protein, 4 aa, Artificial
  synthetic peptide
EEEE

(19) United States
(12) Patent Application Publication
(10) Pub. No.: US 2010/0035824 A1
(43) Pub. Date: Feb. 11, 2010

(54) Synergistic Pharmaceutical Composition

(76) Inventor: Herbert Görne, Hamberg (DE)

Correspondence Address: BUCHANAN INGERSOLL & ROONEY PC, P.O. BOX 1404, ALEXANDRIA, VA, 22313-1404, US

(21) Appl. No.: 12/089,933

(22) PCT Filed: Oct. 10, 2006
(86) PCT No.: PCT/DE2006/001795

§ 371 Date: Aug. 19, 2008

(30) Foreign Application Priority Data
Oct. 12, 2005 (DE) 10 2005 049 372.6

(51) Int. Cl. A61K 38/07; A61K 38/05; A61K 38/06; A61K 38/08; A61P 35/00

(52) U.S. Cl. 514/17; 514/19; 514/18

(57) ABSTRACT

The invention relates to pharmaceutical compositions containing an isoflavone or isoflavone glycoside or a pharmaceutically acceptable salt or solvate of said isoflavone or isoflavone glycoside as an active substance. Also disclosed are uses of such pharmaceutical compositions.

The present invention among others relates to pharmaceutical compositions containing a composition of active substances of metallic ions and/or isoflavones or isoflavone-glycosides and peptides or peptide derivates as well as to the use of such pharmaceutical compositions. Furthermore, this invention relates to the use of selected peptides with a length of 2 to 5 amino acids for the improvement of the availability of active substances in mammals.

Isoflavones which are occasionally also called isoflavonoids form a group of mostly yellow-coloured plant pigments which are derived from isoflavone. Within the frame of this invention, the isoflavones also include their respective glycosides. The most well-known isoflavones in particular are isoflavone, daidzein, genistein, prunetin, biochanin A, orobol, santal, glycitein, pratensein, formononetin, genistin, 6″-O- malonylgenistin, 6″-O-acetylgenistin, daidzin, 6″-O-malonyldaidzin, 6″-O-acetyidaidzin, glycitin, ononin and sissotrin and also genistin, daidzin, 6″-O-malonylglycitin and 6″-O-acetylglycitin. The malonyl-glycosides of the genistein make up the majority of the isoflavones in soybeans. In fermented soy products, the isoflavones are mainly present in their respective aglycon form genistein, daidzein and glycitein.

Some isoflavones are known for having an estrogen effect, for example on grazing animals. Some isoflavones are supposed to have an antioxidant effect within humans or other mammals but such effect could not be proven until now. It is also controversial whether isoflavones have an anti-carcinogenic, anti-atherogenic, anti-osteoporotic and/or hypolipidemic effect and if yes, which ones do. In many cases it has however not been possible until now to reproduce the effect attributed to the isoflavones by administering pure isoflavone, if necessary with the common pharmaceutical carrier and auxiliary substances. In particular it was not possible to achieve an antioxidant effect of genistein despite the administration of high doses and the proof of a high concentration of genistein in the target cells of a human being

However, a similar problem occurs with other pharmaceutical active substances. It is especially known that under certain circumstances, and in particular with diseases, iron is only insufficiently taken in from nourishments by mammals and made available to the mammal, namely the human being. In addition to this, conventional iron compounds are often accompanied by partially severe side-effects. Especially gastrointestinal side-effects are reported.

The task of the present invention therefore was to remedy the above mentioned disadvantages or to at least alleviate them. In particular, compositions supporting or even just initiating the effects of iron and/or isoflavones on mammals should be indicated. As far as possible, therapeutic fields of application of iron and/or the isoflavones should be opened. Another task of the invention was to indicate compositions which improve the availability of pharmaceutical active substances in mammals.

In accordance with the invention, a pharmaceutical composition containing the following components is thus provided:

a combination of active substances including
at least one isoflavone or isoflavone-glycoside and/or pharmaceutically acceptable salts or solvates thereof and
at least one peptide or peptide derivate with 2 to 5 amino acids and/or pharmaceutically acceptable salts or solvates thereof,
   whereupon at least 2 amino acids dispose of side chains which are negatively loaded with pH=7.

In preferred versions, the isoflavone or the isoflavone-glycoside is a substance of the general formula (I) or a pharmaceutically acceptable salt or solvate of such a substance:

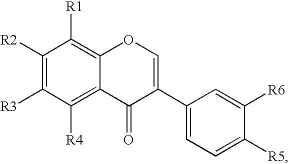

whereupon the residuals R1, R2, R3, R4, R5 and R6 can autonomously have the following meanings: hydrogen, hydroxy, methoxy or glycoside (Glc):

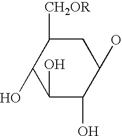

whereupon R is selected from the group consisting of hydrogen, acetyl and malonyl independent of R1, R2, R3, R4, R5 and R6.

Especially preferred are those pharmaceuticals compositions in accordance with the invention in which the active substance is selected from the group consisting of isoflavone, daidzein, genistein, prunetin, biochanin A, orobol, santal, glycitein, pratensein, formononetin, geinstin, 6″-O-malonylgenistin, 6″-O-acetylgenistin, daidzin, 6″-O-malonyidaidzin, 6″-O-acetyldaidzin, glycitin, ononin, sissotrin and mixtures of two or more of these substances.

| Trivial Name | R1 | R2 | R3 | R4 | R5 | R6 | R |
|---|---|---|---|---|---|---|---|
| Isoflavone | H | H | H | H | H | H | — |
| Daidzein | H | OH | H | H | OH | H | — |
| Genistein | H | OH | H | OH | OH | H | — |
| Prunetin | H | OCH₃ | H | OH | OH | H | — |
| Biochanin A | H | OH | H | OH | OCH₃ | H | — |
| Orobol | H | OH | H | OH | OH | OH | — |
| Santal | H | OCH₃ | H | OH | OH | OH | — |
| Glycitein | H | OH | OCH₃ | H | OH | H | — |
| Pratensein | H | OH | H | OH | OCH₃ | OH | — |
| Formononetin | H | OH | H | H | OCH₃ | H | — |
| Genistin | H | Glc | H | OH | OH | H | H |
| 6″-O- | H | Glc | H | OH | OH | H | COCH₂COOH |
| malonylgenistin | | | | | | | |
| 6″-O-acetylgenistin | H | Glc | H | OH | OH | H | COCH₃ |
| Daidzin | H | Glc | H | H | OH | H | H |
| 6″-O- | H | Glc | H | H | OH | H | COCH₂COOH |
| malonyldaidzin | | | | | | | |
| 6″-O-acetyldaidzin | H | Glc | H | H | OH | H | COCH₃ |
| Glycitin | H | Glc | OCH₃ | H | OH | H | H |
| Ononin | H | Glc | H | H | OCH₃ | H | H |
| Sissotrin | H | Glc | H | OH | OCH₃ | H | H |

In other preferred versions, the isoflavone contains 2 to 4 OH groups which are free of or completely or partially substituted by monosaccharides, including monosaccharides which are partially acylated with acetic acid, malonic acid, cinnamic acid, coumaric acid, caffeic acid or ferulic acid, or by disaccharides, including disaccharides which are partially acylated with acetic acid, malonic acid, cinnamic acid, coumaric acid, caffeic acid or ferulic acid, or by methyl or sulphate.

In other preferred versions, the isoflavone or the isoflavone-glycoside is selected from the group consisting of isoflavone, daidzein, genistein, prunetin, biochanin A, orobol, santal, glycitein, pratensein, formononetin, genistin, 6′-O-malonylgenistin, 6″-O-acetylgenistin, daidzin, 6″-O-malonyldaidzin, 6′-O-acetyldaidzin, glycitin, ononin and sissotrin.

In an especially preferred version, the isoflavone is genistein, genistin, daidzein and/or daidzin, whereupon genistein is preferred.

In another especially preferred version, the isoflavone is genistein and the peptide or peptide derivate has the sequence EE, whereupon E=glutamic acid.

The present invention furthermore includes the use of the named combinations of substances containing isoflavone or isoflavone derivates for the production of pharmaceutical compositions for the treatment of and/or the protection against medical indications of the human organism or other mammals' organisms.

With this, preferred medical indications include cardiovascular diseases, diseases connected with an increased thrombocyte aggregation, metabolic disorders or cancer diseases. Especially preferred medical indications include hypertonia, hypercholesterolaemia, heart attack, arteriosclerotic angiopathy, apoplexy, diseases caused by increased thrombocyte aggregation, diabetes mellitus, hyperhomocysteinaemia, malignant tumors and/or osteoporosis.

The present invention furthermore also relates to pharmaceutical compositions including combinations of active substances which contain the following components:
at least one mineral salt, preferably an iron salt, and
at least one peptide or peptide derivate with 2 to 5 amino acids and/or pharmaceutically acceptable salts or solvates thereof,
   whereupon at least 2 amino acids show side chains which are negatively loaded with pH=7.

The present invention moreover relates to the use of the mentioned combinations of active substance containing mineral salts for the production of pharmaceutical compositions for the treatment of and/or the protection against medical indications of the human organism or other mammals' organisms.

With this, preferred medical indications include diseases related to a lack of and/or the increased consumption of the respective mineral as well as pathological or non-pathological loss of blood, e.g. due to gastric/intestinal bleeding.

In addition to this, the present invention includes the use of a composition which contains at least one peptide or peptide derivate and/or pharmaceutically acceptable salts or solvates thereof,
whereupon at least one peptide comprises 2 to 5 amino acids, and
whereupon at least 2 amino acids dispose of side chains which are negatively loaded with pH=7,
   as an additive to pharmaceutical compositions in order to improve the availability of such active substances in these pharmaceutical compositions in mammals.

In preferred versions, the peptide or peptide derivate contains 3 to 5 amino acids, whereupon at least 3 amino acids dispose of side chains which are negatively loaded with pH=7

In preferred versions, the peptide or peptide derivate is a dipeptide which is selected from the group of peptides with the sequences DD, EE, DE, ED, whereupon D=aspartic acid and E=glutamic acid. Such short peptides and their pharmaceutically acceptable salts or solvates have proven to have a strongly synergetic effect, in particular in combination with genistein, genistin, daidzein and daidzin. Of the named dipeptides, glutamic acid dipeptide (a peptide of the sequence EE) is especially preferred. With glutamic acid dipeptide, an extremely remarkable increase of the thrombocyte aggregation inhibiting effect of genistein could be observed with humans in vitro and in vivo. Moreover, the availability of iron for humans could be improved with glutamic acid dipeptide. Correspondingly, especially a pharmaceutical composition in accordance with the invention containing iron and/or genistein, genistin, 6″-O-malonylgenistin and/or 6″-O-acetylgenistin and glutamic acid dipeptide as the active substance is preferred, whereupon however the quantity of other active substances and/or the quantity of glutamic acid dipeptide alone respectively are not therapeutically effective or not to a sufficient extent, but the overall quantity of the other active substances and glutamic acid dipeptide is however therapeutically effective

In other preferred versions the peptide or the peptide derivate is selected from the group of peptides with the sequences

DDDDD, DDDDE, DDDED, DDDEE, DDEDD, DDEDE, DDEED,
DDEEE, DEDDD, DEDDE, DEDED, DEDEE, DEEDD, DEEDE,
DEEED, DEEEE, EDDDD, EDDDE, EDDED, EDDEE, EDEDD,
EDEDE, EDEED, EDEEE, EEDDD, EEDDE, EEDED, EEDEE,
EEEDD, EEEDE, EEEED, EEEEE, DDDD, DDDE, DDED,
DDEE, DEDD, DEDE, DEED, DEEE, EDDD, EDDE, EDED,
EDEE, EEDD, EEDE, EEED, EEEE, DDD, DDE, DED, DEE,
EDD, EDE, EED, EEE;
whereupon D = aspartic acid and E = glutamic acid.

The present invention also includes the use of the named compositions for the reduction of thrombocyte aggregation as a nutritional additive, as an additive for the protection of cells in fermenters or bioreactors, as a nourishment for animals, as a pesticide.

Furthermore, the present invention includes procedures for the production of medicine, especially for the inhibition of thrombocyte aggregation, comprising of the following steps:
a) provision of an active substance selected from the group which consists of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin, and/or a pharmaceutically acceptable solvate or salt of the active substance,
b) provision of a peptide or peptide derivate with a length of 2 to 5 amino acids and/or a solvate or salt of the peptide, whereupon at least two amino acids dispose of a side chain which is negatively loaded with pH=7,
c) mixing of the active substance and/or its solvate or salt with the peptide or peptide derivate and/or its solvate or salt and with a pharmaceutically acceptable carrier.

The starting point of the present invention is the surprising finding that short peptides with a length of 2, 3, 4 or 5 amino acids of which at least two amino acids dispose of a side group which is negatively loaded with pH=7 improve the availability of active substances and especially of iron and isoflavones and isoflavone-glycosides.

In particular, pharmaceutical compositions according to this invention make it possible to reach commonly known effects of a pharmaceutical active substance with the administration of lower quantities to a treated mammal—especially a human being—than those quantities of the corresponding pharmaceutical compositions without the named peptide with a length of 2 to 5 amino acids. Therefore, for the first time it is possible with the compositions according to the invention to achieve pharmaceutical effects of an active substance that has actually been known as such which were not known until now or could not be statistically proven. In particular, the named peptides can synergistically interact with iron and/or isoflavones in order to increase their effects which were already known or supposed in comparison to the effects of pure iron and/or isoflavone. In this, it is recommended that the composition contains the active substance in a quantity which is pharmaceutically effective when the composition is administered to a mammal to be treated. As mentioned before, the pharmaceutically effective quantity can be lower than in comparable peptide-free pharmaceutical compositions due to the presence of the peptide.

In accordance with the invention, active substance relates to a substance which might cause a pharmaceutically desired change of the physiological state of the treated mammal, and especially a human being, when it is administered. Active substance especially relates to the pharmaceutically effective substance of a medicine. If substances are named in their singular form within the frame of this invention, this also relates to the mixture of several of the respective substances if nothing else is specified. Therefore, the invention also concerns pharmaceutical compositions the active substance of which is a mixture of two or several substances and/or the peptide of which is a mixture of two or several peptides. Moreover, according to the invention, an active substance and a peptide also include their respective pharmaceutically acceptable salts or solvates.

Within the frame of the present invention, the term peptide relates to linear or branched peptides which can consist of the 20 gencoded amino acids as well as of the non-naturally appearing alpha, beta and gamma amino acids. Within the frame of the present invention, peptide derivate relates to a peptide which is modified by at least one linear, cyclic or branched alkyl, alkyl ether, alkylthioether, alkoxy, acyl or aryl residue which is substituted by halogen, hydroxyl or amine or non-substituted, saturated or aliphatic in the main and/or the side chain, whereupon the residue contains between 1 and 20 carbon atoms.

It is currently supposed that the peptides applied in accordance with the invention allow for a transport of active substances, especially of iron and isoflavones through cell membranes and thus increase the availability of the active substances and especially of genistein in mammals' target cells. The invention is however not restricted to the accomplishment of this aim.

The pharmaceutical and/or therapeutic effect reached with the active substance and the peptide especially corresponds to the effect of an antioxidant if the active substance is an isoflavone or an isoflavone-glycoside. The pharmaceutical composition in accordance with the invention for the first time makes it possible to achieve the antioxidant effect of an isoflavone which has only been supposed or described with high concentrations of the active substance until now with pharmaceutically acceptable concentrations in the target cells of a treated mammal, especially a human being. Due to the pharmaceutical composition according to the invention, for the first time, a medication is provided with which the supportive effects of isoflavones and isoflavone-glycosides which were only supposed until now can be reproducibly achieved. Isoflavones, isoflavone-glycosides and their pharmaceutically acceptable salts or solvates can thus for the first time be used with a known, equal and reproducible structure in a pharmaceutical composition.

It is especially preferred if the quantity of the active substance and/or the quantity of the peptide alone respectively is not sufficient for the generation of the pharmaceutical and/or therapeutic effect.

A composition in accordance with the invention is especially preferred to one of the previously described ways of reducing the availability of hydrogen peroxide in a mammal, and especially a human being. The active substance of this composition expediently is an isoflavone and/or isoflavone-glycoside. Hydrogen peroxide is an initiating or at least supportive factor for the development of numerous diseases of mammals and especially humans. It has not been possible to reduce the availability of hydrogen peroxide and to eliminate its disease-supporting or disease-inducing effect with the common isoflavone compositions, especially not with human beings. It was especially not possible until now to reproducibly reduce the availability of hydrogen peroxide in the body cells of humans or other mammals in a physiologically effective way by administering especially the isoflavones and isoflavone-glycosides genistein, daidzein, genistin and/or daidzin. The pharmaceutical composition according to the invention remedies this for the first time.

Furthermore, a composition in accordance with the invention is preferred to one of the ways described before which aims at the inhibition of thrombocyte aggregation, the prevention and/or treatment of hypertonia, hypercholesterolaemia, hyperhomocysteinaemia, diabetes mellitus, heart attack, apoplexy, arteriosclerotic angiopathy, malignant tumors and osteoporosis, whereupon it is again appropriate to provide an isoflavone and/or isoflavone-glycoside as the active substance.

Preferably, the active substance(s) and peptide(s) but not the respective individual quantities of the active substance(s) and/or peptide(s) in the pharmaceutical composition according to the invention are given in a quantity which is sufficient in order to cause an inhibition of thrombocyte aggregation when it is administered to a mammal and especially a human being. Such an effect could not reproducibly be reached with the administration of daidzein, daidzin, genistein and/or genistin, if necessary with conventional pharmaceutical auxiliaries and carriers with the use of physiologically acceptable concentrations of said active substance and/or said active substances. Therefore, the pharmaceutical composition in accordance with the invention is advantageously suited as a substitute of conventional pharmaceutical compositions containing acetylsalicylic acid and/or clopidogrel. The pharmaceutical compositions according to the invention make it possible to reach a desired therapeutic effect without the known side-effects of conventional pharmaceutical compositions with active substances based on acetylsalicylic acid and/or clopidogrel, and especially to inhibit the thrombocyte aggregation in the blood of a treated person or another mammal.

With the said substances, especially with genistein, it is possible to produce pharmaceutical compositions in accordance with the invention which are particularly suited for the inhibition of thrombocyte aggregation in a mammal as for example a human. With such compositions in accordance with the invention, in particular the undesired side-effects and treatment failures which occur in connection with the use of conventional thrombocyte aggregation inhibitors based on acetylsalicylic acid and/or clopidogrel can be prevented or at least reduced.

The therapeutic effectiveness can especially include or consist of an antioxidant effect and especially the reduction of the availability of hydrogen peroxide in a mammal, and preferably the inhibition of thrombocyte aggregation.

The pharmaceutical preparation in accordance with the invention is preferably made for oral or parenteral application. For this, the pharmaceutical composition in accordance with the invention can be given in the form of a tablet, dragêe, juice or another solution. The pharmaceutical composition in accordance with the invention can preferably contain the following pharmaceutically acceptable carriers and auxiliaries: water and glucose. The expert will find further carriers and auxiliaries listed in the publication of Fiedler, H. P., Lexikon der Hilfsstoffe für Pharmazie, Kosmetik und angrenzende Gebiete [Dictionary of auxiliaries for pharmaceuticals and cosmetics and related fields], 4. edition, Aulendorf: ECV-Editio-Kantor-Verlag, 1996.

Preferably, the composition in accordance with the invention is formulated as a solid or liquid medicine, especially powder, fine powder, granulate, tablets, especially film-coated tablets, pastilles, sachets, cachets, dragees, capsules, ointments, creams, hydrogels, pastes, patches, solutions, emulsions, especially of the type oil in water, suspensions such as for example lotions, injection and infusion preparations.

Depending on the way of preparation, the pharmaceutical composition in accordance with the invention contains the active substance and the peptide in especially chosen quantities. Usually, a preparation in accordance with the invention will also contain iron in the quantities which are known until now and which might also depend on the way of preparation. If the pharmaceutical composition in accordance with the invention contains an isoflavone and/or isoflavone-glycoside as the active substance, the quantity thereof will be at least 1 mg as per administered unit (e.g. as per tablet) and preferably up to 500 mg as per administered unit. Particularly preferred compositions in accordance with the invention contain a total of 10 mg up to 500 mg of isoflavone(s) and/or isoflavone-glycoside(s) as per administered unit, especially as per tablet, whereupon for the administration in the form of tablets, quantities of 100 mg to 500 mg are preferred. For the administration in a solution to be administered parenterally, the concentration of the isoflavone and/or isoflavone-glycoside active substance(s) amounts to at least 0.1 mg/ml and preferably to up to 100 mg/ml, and particularly preferred 10 to 50 mg/ml.

The peptide and/or the peptides are preferably provided in an overall proportion (peptide/other active substances mol/mol) of 1:2 to 10:1 in relation to the total quantity of other active substances. Preferably, the overall proportion (peptide/other active substances mol/mol) is 3:1 to 5:1.

In accordance with the invention, the use of a peptide with a length of 2 to 5 amino acids or of a pharmaceutically acceptable solvate or salt of the peptide is recommended, whereupon at least 2 of the amino acids dispose of a side chain which is negatively loaded with pH 7 in order to improve the availability of an active substance in a mammal, to produce a medicine for the inhibition of thrombocyte aggregation, to prevent and/or treat hypertonia, hypercholesterolaemia, hyperhomocysteinaemia, diabetes mellitus, heart attack, apoplexy, arteriosclerotic angiopathy, malignant tumors and/or osteoporosis. Expediently, the peptide and/or the peptide mixture is used together with an isoflavone and/or isoflavone-glycoside or a mixture of those substances in order to produce a medicine for the prevention and/or treatment of hypertonia, hypercholesterolaemia, hyperhomocysteinaemia, diabetes mellitus, heart attack, apoplexy, arteriosclerotic angiopathy, malignant tumors and/or osteoporosis. Such use allows for the beneficially simple exploitation of the advantages described above of the pharmaceutical composition in accordance with the invention, and especially of the preferred version thereof which has been described in more detail above.

With this, the preference is for a use where the quantity of the active substance(s) and/or the quantity of peptide(s) alone respectively is not therapeutically effective in the medicine but the total quantity of the active substance(s) and peptide(s) is therapeutically effective. According to this, the preference is for a use where the quantity and/or concentration of the peptide or the pharmaceutically acceptable solvate or salt of the peptide is sufficient in order to improve the availability of an active substance inducing the therapeutic effect of the active substance in a mammal that is treated with the medicine.

Furthermore, a procedure for the production of a medicine which especially serves the inhibition of thrombocyte aggregation consisting of the following steps is provided.
a) provision of an active substance selected from the group consisting of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin, and/or a pharmaceutically acceptable solvate or salt of the active substance,
b) provision of a peptide with a length of up to 5 amino acids and/or a solvate or salt of the peptide, whereupon at least two of the amino acids dispose of a side chain which is negatively loaded with pH 7,
c) mixing of the active substance and/or its solvate or salt with the peptide and/or its solvate or salt and with a pharmaceutically acceptable carrier.
   The invention will be further explained with the following examples and figures, whereupon the object of the invention is not restricted to those examples and figures. The following is shown:

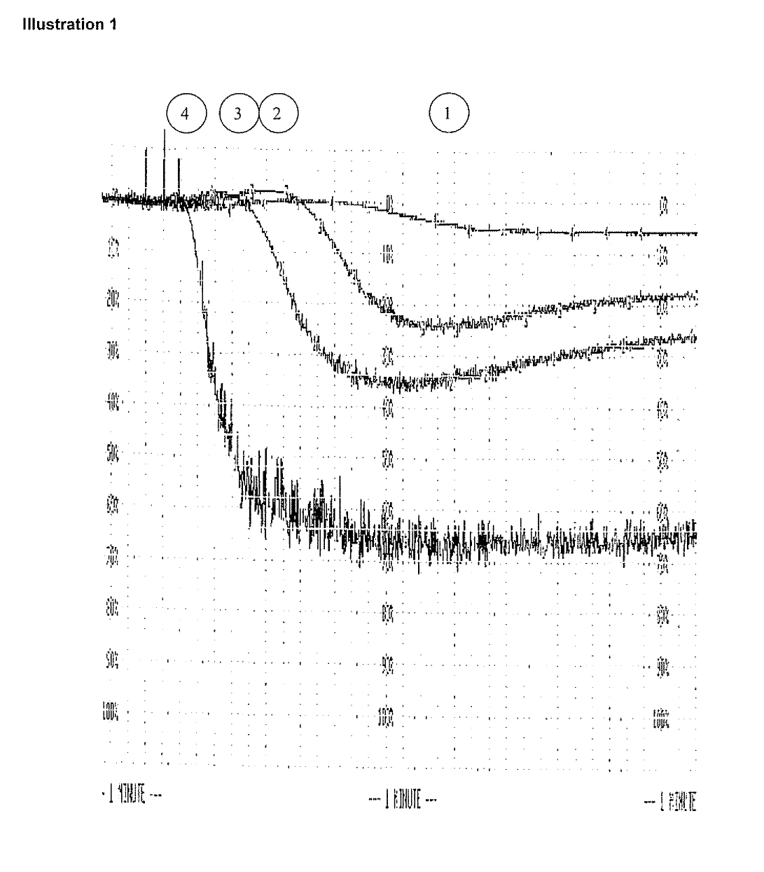

FIG. 1 Presentation of the inhibition of the collagen-induced thrombocyte aggregation of human blood plasma which is rich of platelets with the effect of different genistein concentrations,

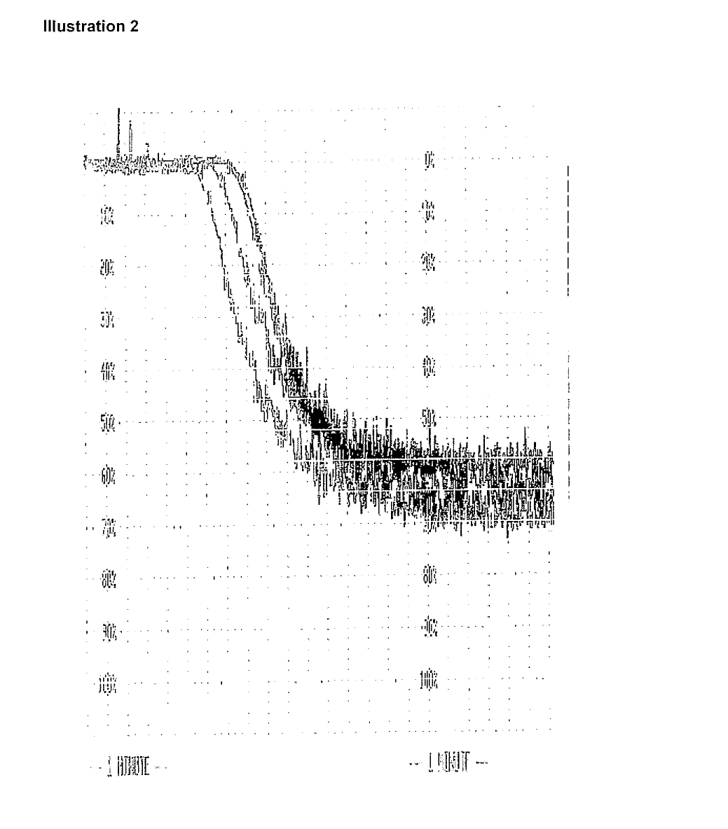

FIG. 2 Presentation of the lacking effect of the glutamic acid dipeptide on the collagen-induced thrombocyte aggregation of human blood plasma which is rich of platelets,

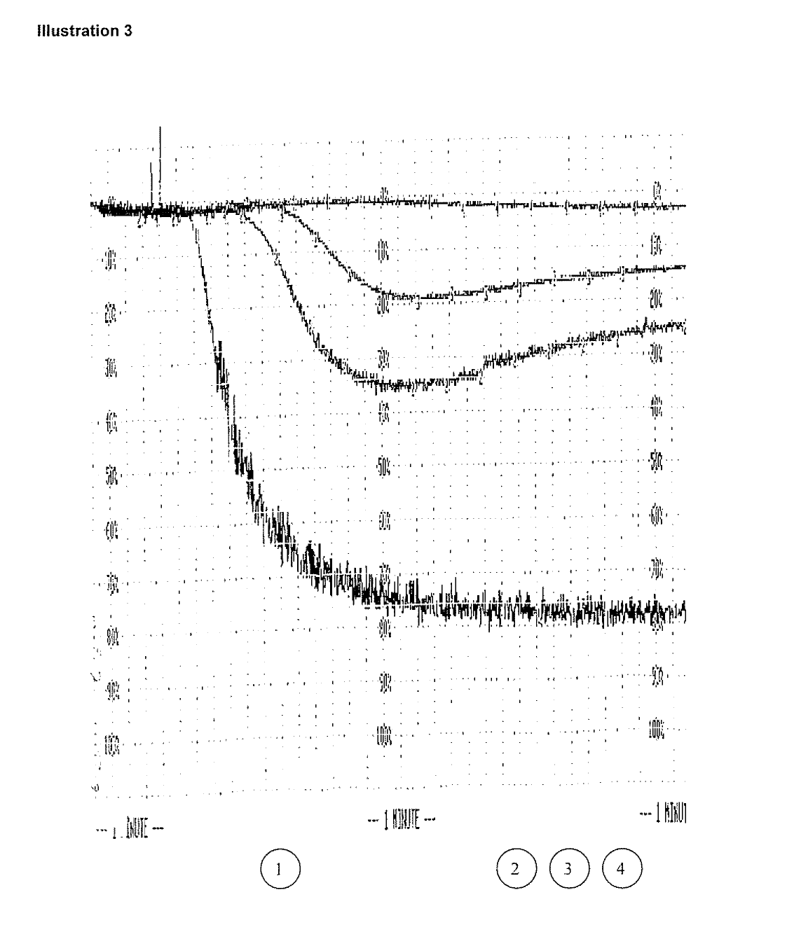

FIG. 3 Impact of different concentrations of the glutamic acid dipeptide on the inhibition of the collagen-induced thrombocyte aggregation of genistein,

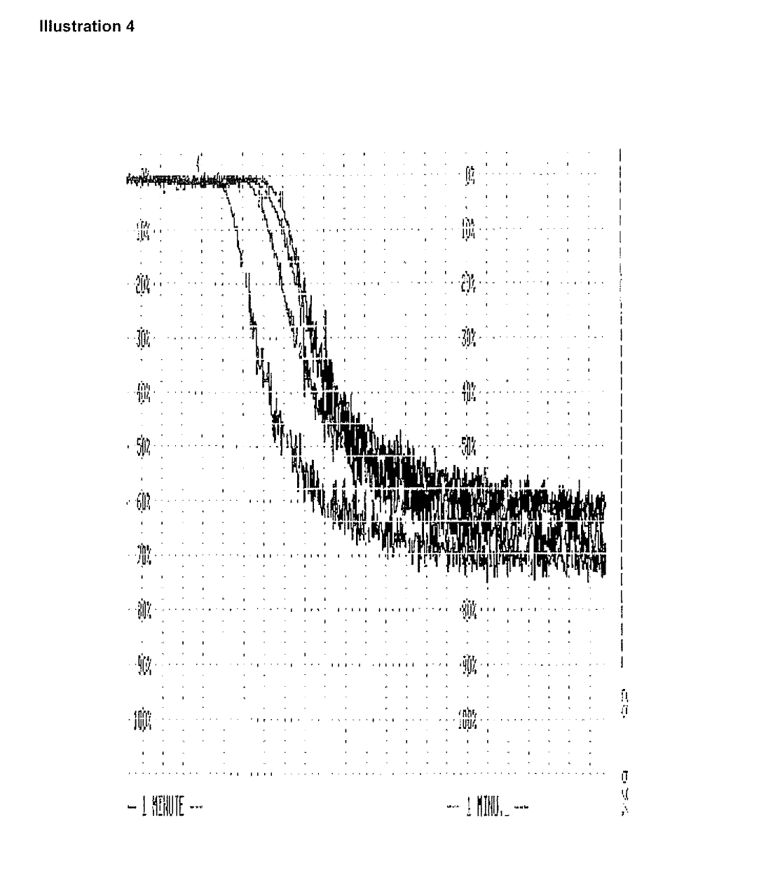

FIG. 4 Presentation of the lacking effect of alanine dipeptide on the genistein-induced inhibition of thrombocyte aggregation, and

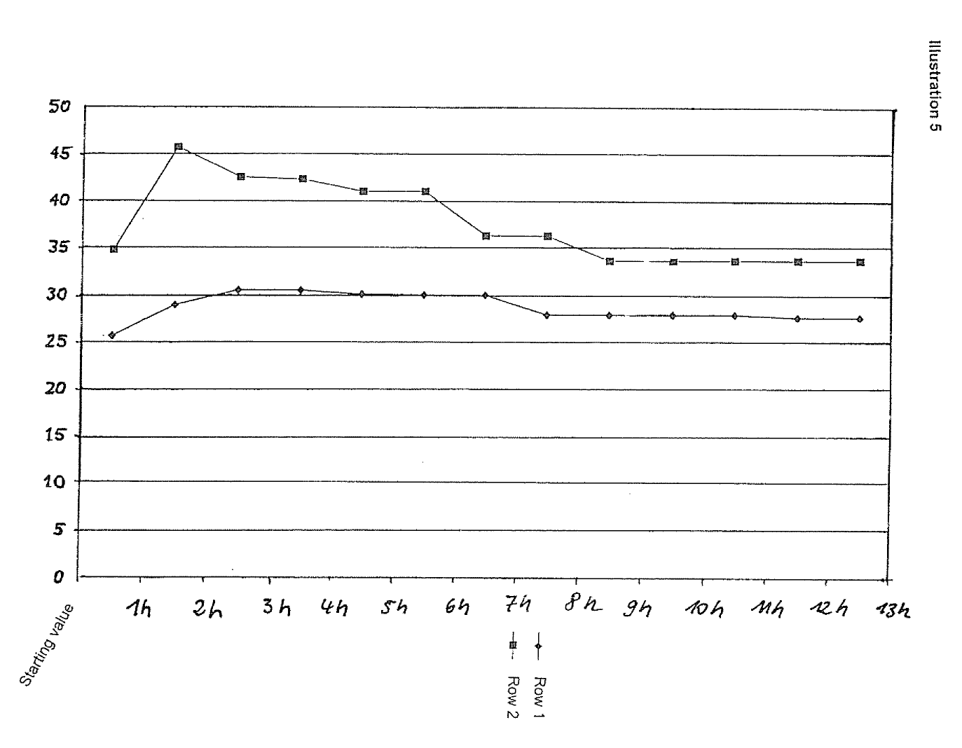

FIG. 5 Presentation of the concentration of iron in the serum (mg Fe²⁺/ml blood serum) with and without the administration of glutamic acid dipeptide.

### EXAMPLE 1

### Inhibition of the Collagen-Induced Thrombocyte Aggregation by Genistein in Vitro

FIG. 1 shows the extent of the thrombocyte aggregation after the induction of collagen (U. Budde, Diagnose von Funktionsstörungen der Thrombozyten mit Hilfe der Aggregometrie [Diagnosis of functional disorders of thrombocytes with aggregometry], J. Lab. Med. 2002, 26(11/12), 564-571) with different genistein concentrations. In the absence of genistein and after the addition of collagen to human plasma which is rich in platelets, a thrombocyte aggregation of about 65% is reached. After the addition of genistein 100 uM, the thrombocyte aggregation falls to about 35%. With a genistein concentration of 150 uM, the maximum thrombocyte aggregation amounts to about 23%, while with 200 uM of genistein, a maximum thrombocyte aggregation of about 5% is reached.

### EXAMPLE 2

### Lacking Effect of the Glutamic Acid Dipeptide (EE) on the Thrombocyte Aggregation in Vitro

While the other conditions of the experiment remained the same as in Example 1, with a concentration of glutamic acid dipeptide (without genistein) of 100 μmolar, the thrombocyte aggregation fell from 65% to about 60% in comparison to a corresponding control reaction.

### EXAMPLE 3

### Synergistic Effect of Genistein and Glutamic Acid Dipeptide for the Inhibition of Thrombocyte Aggregation

While the other conditions of the experiment remained the same as in Example 1, the collagen-induced thrombocyte aggregation was measured with different concentrations of genistein and glutamic acid dipeptide. In absence of genistein and glutamic acid dipeptide, the results of Example 1 were reproduced. In presence of 50 uM of genistein but in absence of glutamic acid dipeptide, a maximum thrombocyte aggregation of 25% was reached. This roughly corresponds to the result which had to be expected against the background of Example 1. In presence of 50 uM of genistein and 150 uMr of glutamic acid dipeptide, the maximum thrombocyte aggregation reached was reduced to about 15%. Thus, the thrombocyte aggregation inhibiting effect of the mixture of glutamic acid dipeptide and genistein in accordance with the invention is higher than the effect of the pure genistein. With a final concentration of 50 uM of genistein and 250 uM of glutamic acid dipeptide, practically no thrombocyte aggregation was observed. This composition in accordance with the invention is thus more effective for the inhibition of thrombocyte aggregation than genistein in the same concentration.

### EXAMPLE 4

### Use of Alanine Dipeptide Instead of Glutamic Acid Dipeptide

It was tested whether dipeptides which do not dispose of side chains which are negatively loaded with pH 7 have a similar effect as glutamic acid dipeptide. For this, the collagen-induced thrombocyte aggregation was measured with different concentrations of genistein and alanine dipeptide while the other conditions of the experiment remained the same as in Example 1 . FIG. 4 shows that in absence of genistein and alanine dipeptide, a maximum thrombocyte aggregation of about 70% could be caused. With a concentration of 50 uM of genistein, a maximum thrombocyte aggregation of about 60% was observed regardless of whether the concentration of alanine dipeptide was 0 uM, 50 uM or 200 uM.

### EXAMPLE 5

### Effect of Inhibiting Thrombocyte Aggregation of a Composition in Accordance with the Invention in Vitro

A test person orally took in a mixture of genistein, glutamic acid dipeptide and a pharmaceutically acceptable carrier for one week. In this time, it was observed whether there was an inhibition of the thrombocyte aggregation. The following table shows the results:

| Daily dose | Daily dose of glutamic acid | Inhibition of the thrombocyte |
|---|---|---|
| of genistein | dipeptide | aggregation |
| 50 mg | 5 mg | No effect was observed |
| 300 mg | 30 mg | An inhibition of thrombocyte |
| | | aggregation of the Aspirin- |
| | | type was observed |

The pharmaceutical composition in accordance with the invention is thus able to inhibit the thrombocyte aggregation in a human in vivo.

### EXAMPLE 6

### Improvement of the Availability of Iron

First of all, the iron concentration in the serum of a test person prior to taking a preparation in accordance with the invention and/or a control preparation is measured with conventional procedures (determination of the zero value). In an experiment, the test person then takes in a Vitaferro drops (80 mg Fe++) in 150 ml water as a positive control. The iron concentration in the serum is determined on an hourly basis after the taking (“sample 1” after 1 h, “sample 2” after 2 h etc ; FIG. 5 row 1). In another experiment carried out 14 days later, 100 mg glutamic acid dipeptide are added to the Vitaferro drops (80 mg Fe++) in 150 ml water which is taken after the determination of the zero value. Again, the iron concentration in the serum of the test person is determined on an hourly basis (FIG. 5 row 2) The examination procedure corresponds to the provision of Paschen et al., Prüfung der Bioäquivalenz von zwei schnellfreisetzenden Eisen (II)-Sulfat-Formulierungen [Testing of the bioequivalence of two rapid-release iron (II) sulphate formulae], Arzneim -Forsch./Drug Res. 43(II), no, 11 1993, The addition of 100 mg glutamic acid dipeptide leads to a considerably improved availability of iron (Fe²⁺) in the test person.

### Legend for the Illustrations:

### Illustration 1

Collagen-induced thrombocyte aggregation in presence of genistein in an increasing concentration:

1=0 μM; 2=100 μM; 3=150 μM; 4=200 μM

### Illustration 2

Collagen-induced thrombocyte aggregation with glu-glu in an increasing concentration:

A=0 μM; B=25 μM; C=50 μM; D=100 μM

### Illustration 3

Collagen-induced thrombocyte aggregation with 50 μM of genistein alone, after the addition of glu-glu (150 μM and/or 250 μM) as well as reference

### Illustration 4

Collagen-induced thrombocyte aggregation in presence of genistein 50 μM as well as of ala-ala in an increasing concentration.

B=50 μM/0 μM; C=50 μM/50 μM; D=50 μM/200 μM; A=0 μM/0 μM Reference

1. Pharmaceutical composition with a combination of active substances, containing
at least one isoflavone or isoflavone-glycoside and/or pharmaceutically acceptable salts or solvates thereof and
at least one peptide or peptide derivate with 2 to 5 amino acids and/or pharmaceutically acceptable salts or solvates thereof,
whereupon at least 2 amino acids show side chains which are negatively loaded with pH=7.

2. Composition in accordance with claim 1, whereupon the isoflavone contains 2-4 OH groups which are free of or completely or partially substituted by monosaccharides, including monosaccharides which are partially acylated with acetic acid, malonic acid, cinnamic acid, coumaric acid, caffeic acid or ferulic acid, or by disaccharides, including disaccharides which are partially acylated with acetic acid, malonic acid, cinnamic acid, coumaric acid, caffeic acid or ferulic acid, or by methyl or sulphate.

3. Composition in accordance with claim 1, whereupon the isoflavone or isoflavone-glycoside is selected from the group consisting of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin.

4. Composition in accordance with claim 1, whereupon the isoflavone is genistein.

5. Composition in accordance with claim 1, whereupon the peptide or peptide derivate contains 3 to 5 amino acids and at least 3 amino acids include side chains which are negatively loaded with pH=7.

6. Composition in accordance with claim 1, whereupon the peptide or peptide derivate is a dipeptide which is selected from the group of peptides with the sequences DD, EE, DE, ED, whereupon D=aspartic acid and E=glutamic acid.

7. Composition in accordance with claim 1 to 6, whereupon the peptide or peptide derivate is selected from the group of peptides with the sequences DDDDD (SEQ ID No. 1), DDDDE (SEQ ID No. 2), DDDED (SEQ ID No. 3), DDDEE (SEQ ID No. 4), DDEDD (SEQ ID No. 5), DDEDE (SEQ ID No. 6), DDEED (SEQ ID No. 7), DDEEE (SEQ ID No. 8), DEDDD (SEQ ID No. 9), DEDDE (SEQ ID No. 10), DEDED (SEQ ID No. 11), DEDEE (SEQ ID No. 12), DEEDD (SEQ ID No. 13), DEEDE (SEQ ID No. 14), DEEED (SEQ ID No. 15), DEEEE (SEQ ID No. 16), EDDDD (SEQ ID No. 17), EDDDE (SEQ ID No. 18), EDDED (SEQ ID No. 19), EDDEE (SEQ ID No. 20), EDEDD (SEQ ID No. 21), EDEDE (SEQ ID No. 22), EDEED (SEQ ID No. 23), EDEEE (SEQ ID No. 24), EEDDD (SEQ ID No. 25), EEDDE (SEQ ID No. 26), EEDED (SEQ ID No. 27), EEDEE (SEQ ID No. 28), EEEDD (SEQ ID No. 29), EEEDE (SEQ ID No. 30), EEEED (SEQ ID No. 31), EEEEE (SEQ ID No. 32), DDDD (SEQ ID No. 33), DDDE (SEQ ID No. 34), DDED (SEQ ID No. 35), DDEE (SEQ ID No. 36), DEDD (SEQ ID No. 37), DEDE (SEQ ID No. 38), DEED (SEQ ID No. 39), DEEE (SEQ ID No. 40), EDDD (SEQ ID No. 41), EDDE (SEQ ID No. 42), EDED (SEQ ID No. 43), EDEE (SEQ ID No. 44), EEDD (SEQ ID No. 45), EEDE (SEQ ID No. 46), EEED (SEQ ID No. 47), EEEE (SEQ ID No. 48), DDD, DDE, DED, DEE, EDD, EDE, EED, and EEE; whereupon D=aspartic acid and E=glutamic acid.

8. (canceled)

9. Composition in accordance with claim 1, whereupon the substance proportion Q=nP/nl with nP=substance quantity of the peptide or peptide derivate and nl=substance quantity of isoflavone or isoflavone-glycoside is between 3 and 5.

10. A method of treatment of and/or the protection against a medical indication selected from the group consisting of: cardiovascular diseases, diseases connected with an increased thrombocyte aggregation, metabolic disorders, bone diseases, cancer diseases, hypertonia, hypercholesterolaemia, heart attack, arteriosclerotic angiopathy, apoplexy, diseases caused by increased thrombocyte aggregation, diabetes mellitus, hyperhomocysteinaemia, malignant tumors, and osteoporosis, comprising adminstering an effective amount of a composition of claim 1.

11-14. (canceled)

15. Pharmaceutical composition including a combination of active substances which contains
at least one mineral salt, preferably an iron salt, and
at least one peptide or peptide derivate with 2 to 5 amino acids and/or pharmaceutically acceptable salts or solvates thereof,
whereupon at least 2 amino acids show side chains which are negatively loaded with pH=7.

16. A process for the production of a pharmaceutical composition for the treatment of and/or the protection against diseases which are associated with a lack of and/or an increased consumption of the respective material, preferably iron, comprising providing a dosage form comprising an effective amount of a pharmaceutical composition of claim 15.

17. A process for the production of a pharmaceutical composition for the treatment of pathological and non-pathological loss of blood, such as gastric/intestinal bleeding, comprising providing a dosage form comprising an effective amount of a pharmaceutical composition of claim 15.

18. A pharmaceutical composition of claim 10, wherein the peptide or peptide derivate contains 3 to 5 amino acids and whereupon at least 3 amino acids include side chains which are negatively loaded with pH=7.

19. A pharmaceutical composition of claim 10, wherein the peptide or peptide derivate is selected from the group of peptides with the sequences

| | |
|---|---|
| DDDDD, | (SEQ ID No. 1) |
| DDDDE, | (SEQ ID No. 2) |
| DDDED, | (SEQ ID No. 3) |
| DDDEE, | (SEQ ID No. 4) |
| DDEDD, | (SEQ ID No. 5) |
| DDEDE, | (SEQ ID No. 6) |
| DDEED, | (SEQ ID No. 7) |
| DDEEE, | (SEQ ID No. 8) |
| DEDDD, | (SEQ ID No. 9) |
| DEDDE, | (SEQ ID No. 10) |
| DEDED, | (SEQ ID No. 11) |
| DEDEE, | (SEQ ID No. 12) |
| DEEDD, | (SEQ ID No. 13) |
| DEEDE, | (SEQ ID No. 14) |
| DEEED, | (SEQ ID No. 15) |
| DEEEE, | (SEQ ID No. 16) |
| EDDDD, | (SEQ ID No. 17) |
| EDDDE, | (SEQ ID No. 18) |
| EDDED, | (SEQ ID No. 19) |
| EDDEE, | (SEQ ID No. 20) |
| EDEDD, | (SEQ ID No. 21) |
| EDEDE, | (SEQ ID No. 22) |
| EDEED, | (SEQ ID No. 23) |
| EDEEE, | (SEQ ID No. 24) |
| EEDDD, | (SEQ ID No. 25) |
| EEDDE, | (SEQ ID No. 26) |
| EEDED, | (SEQ ID No. 27) |
| EEDEE, | (SEQ ID No. 28) |
| EEEDD, | (SEQ ID No. 29) |
| EEEDE, | (SEQ ID No. 30) |
| EEEED, | (SEQ ID No. 31) |
| EEEEE, | (SEQ ID No. 32) |
| DDDD, | (SEQ ID No. 33) |
| DDDE, | (SEQ ID No. 34) |
| DDED, | (SEQ ID No. 35) |
| DDEE, | (SEQ ID No. 36) |
| DEDD, | (SEQ ID No. 37) |
| DEDE, | (SEQ ID No. 38) |
| DEED, | (SEQ ID No. 39) |
| DEEE, | (SEQ ID No. 40) |
| EDDD, | (SEQ ID No. 41) |
| EDDE, | (SEQ ID No. 42) |
| EDED, | (SEQ ID No. 43) |
| EDEE, | (SEQ ID No. 44) |
| EEDD, | (SEQ ID No. 45) |
| EEDE, | (SEQ ID No. 46) |
| EEED, | (SEQ ID No. 47) |
| EEEE, | (SEQ ID No. 48) |

DDD, DDE, DED, DEE, EDD, EDE, EED,
EEE, DD, EE, DE, ED,
whereupon D = aspartic acid and E = glutamic acid.

20. The pharmaceutical composition of claim 10, wherein the isoflavone is genistein and the peptide or peptide derivate has the sequence EE, whereupon E=glutamic acid.

21. A method of providing a pharmaceutical composition for the reduction of an increased thrombocyte aggregation, comprising providing a dosage form comprising a therapeutically effective amount of the composition of claim 10.

22. A process for providing at least one member of the group consisting of a nutritional additive, an additive for the protection of cells in fermenters and bioreactors, nourishment for animals, and a pesticide, comprising providing a composition of claim 1.

23-25. (canceled)

26. A process for providing an additive for pharmaceutical compositions to increase the availability of active substances in such pharmaceutical compositions in mammals, comprising providing to a mammal in need of available active substances a composition of claim 1.

27-28. (canceled)

29. Procedure for the production of a medicine, especially for the inhibition of thrombocyte aggregation, comprising of the following steps:
(a) provision of an active substance selected from the group which consists of isoflavone, daidzein, genistein, prunetin, biochanin A, orobol, santal, glycitein, pratensein, formononetin, genistin, 6″-O-malonylgenistin, 6″-O-acetylgenistin, daidzin, 6″-O-malonyldaidzin, 6″-O-acetyldaidzin, glycitin, ononin and sissotrin, and/or a pharmaceutically acceptable solvate or salt of the active substance,
(b) provision of a peptide or peptide derivate with a length of 2 to 5 amino acids and/or a solvate or salt of the peptide, whereupon at least two of the amino acids have to dispose of a sidechain which is negatively loaded with pH=7,
(c) mixing of the active substance and/or its solvate or salt with the peptide or peptide derivate and/or its solvate or salt and with a pharmaceutically acceptable carrier.
